(19) United States
(12) Patent Application Publication
(10) Pub. No.: US 2026/0083626 A1
(43) Pub. Date: Mar. 26, 2026

(54) MASSAGE MACHINE

(71) Applicant: FUJI MEDICAL INSTRUMENTS MFG. CO., LTD., Osaka (JP)

(72) Inventors: Kentaro ODE, Osaka (JP); Yoshinori ONO, Osaka (JP)

(73) Assignee: FUJI MEDICAL INSTRUMENTS MFG. CO., LTD., Osaka (JP)

(21) Appl. No.: 19/333,884

(22) Filed: Sep. 19, 2025

(30) Foreign Application Priority Data
Sep. 26, 2024 (JP) 2024-167352
Jun. 9, 2025 (JP) 2025-095633

(51) Int. Cl. A61H 15/00; A61H 7/00; A61H 9/00; G16H 40/63

(52) U.S. Cl. CPC A61H 15/0078; A61H 7/007; A61H 9/0078; G16H 40/63; A61H 2015/0035; A61H 2201/0149; A61H 2201/5005; A61H 2201/5007; A61H 2201/5043; A61H 2201/5071; A61H 2205/081

(57) ABSTRACT

Provided is a massage machine including: a treatment part including a lateral side abutment body that is movable in a width direction, abuts on a lateral side of the body of a user, and treats the lateral side of the body of the user; a detector that detects abutment of the lateral side abutment body with the body of the user by movement of the lateral side abutment body; and a controller that determines a treatment intensity based on a detection result of the detector and actuates the treatment part at the determined treatment intensity.

### CROSS-REFERENCE TO RELATED APPLICATION

This application claims priority to Japanese Patent Application No. 2024-167352, filed on Sep. 26, 2024, and Japanese Patent Application No. 2025-095633, filed on Jun. 9, 2025, both of which are incorporated by reference in their entireties.

### FIELD

The present disclosure relates to a massage machine.

### BACKGROUND

There exist massage machines, which include a seat and a backrest. Among these, there is a massage machine that estimates the body type of a person to be treated (i.e., user) (for example, JP 2021-94156 A). In the massage machine described in the above document, a mat-shaped pressure measuring part is provided on the backrest. When the user leans against the backrest, the pressure measuring part deforms in accordance with the body type of the user. A controller estimates the body type of the user based on the pressure applied to the pressure measuring part.

However, the invention described in the above document aims to start treatment quickly by quickly estimating the body type before the start of treatment, that is, to shorten the waiting time of the user. For this reason, the above-mentioned document does not disclose any specific control content based on the estimated body type, and the invention described in the above-mentioned document has room for improvement in terms of utilizing the result of the estimation of the body type in the treatment content. Further, depending on the posture of the user, it may be difficult to continue appropriate treatment, or treatment may not be performed in a predetermined order (treatment course). The invention described in the above document does not sufficiently consider these points.

### SUMMARY

It is therefore an object of the present disclosure to provide a massage machine that can provide a treatment that is suited to the physical conditions of the user.

The following presents a simplified summary of the disclosure disclosed herein in order to provide a basic understanding of some aspects of the disclosure. This summary is not an extensive overview of the disclosure. It is intended to neither identify key or critical elements of the disclosure nor delineate the scope of the disclosure. Its sole purpose is to present some basic concepts of the disclosure in a simplified form as a prelude to the more detailed description that is presented later.

As an example, a massage machine of the present disclosure includes: a treatment part including a lateral side abutment body that is movable in a width direction, abuts on a lateral side of the body of a user, and treats the lateral side of the body of the user; a detector that detects abutment of the lateral side abutment body with the body of the user by movement of the lateral side abutment body; and a controller that determines a treatment intensity based on a detection result of the detector and actuates the treatment part at the determined treatment intensity.

### BRIEF DESCRIPTION OF DRAWINGS

The foregoing and other features of the present disclosure will become apparent from the following detailed description and drawings of an illustrated embodiment of the present disclosure.

FIG. 1 is a perspective view schematically showing a massage machine according to an embodiment of the present disclosure.

FIG. 2 is a schematic view showing the arrangement of a plurality of airbags with respect to the user and a movement of a back side treatment part (particularly treatment elements) in the massage machine.

FIG. 3 is a perspective view showing an example of the configuration of an internal mechanism of the back side treatment part provided in the massage machine.

FIG. 4 is a block diagram showing the configuration of the massage machine relating to the airbag.

FIG. 5 is a flow diagram relating to the body type detection in the massage machine.

FIG. 6 is a flow diagram showing the control of the airbags in the massage machine according to the result of the body type detection.

FIG. 7A is a schematic diagram showing how the treatment range of the back side treatment part in the massage machine is changed, showing a case where the treatment range is large in the width direction.

FIG. 7B is a schematic diagram showing how the treatment range of the back side treatment part is changed in the massage machine, showing a case where the treatment range is small in the width direction.

FIG. 8 is a graph (specific numerical values omitted) showing an example of the change in pressure over time for each body type of the user in the massage machine. The line types are differentiated depending on whether the user is not seated (“no body”) or whether the body width is relatively “small,” “medium,” or “large.”

### DETAILED DESCRIPTION OF THE INVENTION

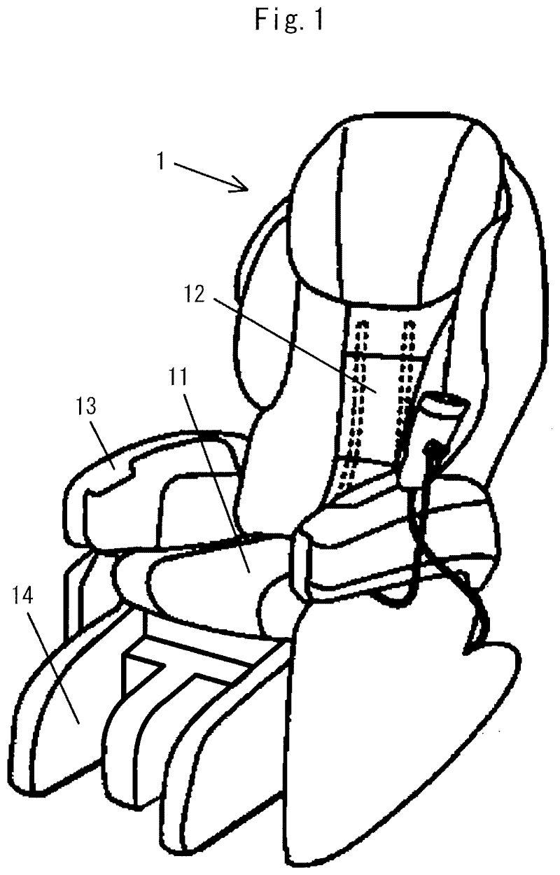

Next, a massage machine 1 according to an embodiment of the present disclosure will be described. The directions hereinafter are expressed as directions in the massage machine (chair-type massage machine) shown in FIG. 1. Further, the “width direction” refers to the left and right direction when viewed from the front or back, and further, is the direction that coincides with the direction along the body width of the user P when he or she is sitting on the massage machine 1. The width direction may also be referred to as the “lateral direction.”

### —Configuration of Massage Machine—

The massage machine 1 of this embodiment is a chair-type massage machine as shown in FIG. 1 (note that it shall not be limited to the illustrated form), and includes a seat 11, a backrest 12, armrests 13, and an ottoman 14. The configuration of each part is basically the same as that of the conventional one, and therefore a detailed description will not be given. The massage machine 1 includes a treatment part 2 for providing treatment. In this embodiment, provided as the treatment part 2 are a back side treatment part 3 that provides treatment to the back of the body of the user P, and an airbag 4 that forms a lateral side treatment part that provides treatment to each of the lateral sides of the body of the user P. The airbag 4 inflates and deflates by an air supply mechanism 5. The airbag 4 is inflated by supplying air thereinto, or is deflated by releasing air therefrom. The massage machine 1 further includes a detector 6 that performs detection for treatment, and a controller 7 that controls each part of the massage machine 1. In particular, the controller 7 controls the actuations of the back side treatment part 3 and the lateral side treatment part (a plurality of airbags 4).

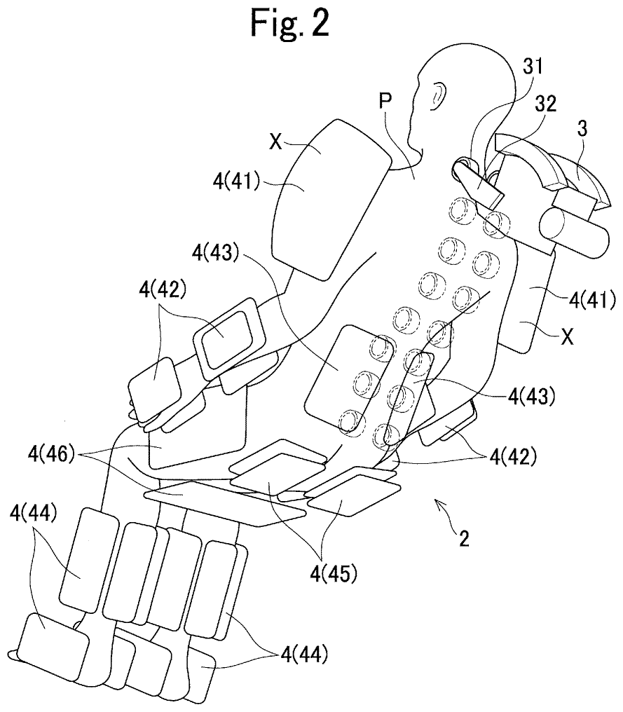

The backside treatment part 3 is disposed inside the backrest 12 located on the back side of the seat 11. The back side treatment part 3 performs treatment on the body of the user P who is sitting on the seat 11 and leaning his/her back against the backrest 12. The back side treatment part 3 moves relative to the body of the user Pin at least one direction among front-back, up-down, and left-right. The back side treatment part 3 is an integral unit as shown in FIG. 2. The back side treatment part 3 of this embodiment performs treatment on the body of the user P facing the backrest 12 by pressing from outside the body of the user P and moving along the body of the user P. It can also be configured such that the range of the movement of the back side treatment part 3 extends to the seat 11 so that treatment can be performed to the buttocks or thighs of the user P. In the back side treatment part 3, pressing is applied to the body in the form of “kneading” or “tapping”, and pressing accompanied by movement is applied to the body in the form of “rubbing (scrubbing)”, “kneading”, or “tapping”. As shown in FIG. 1, a sheet of the backrest 12 (made of cloth or leather) is present between the back side treatment part 3 and the body of the user P, so the pressing is applied through the sheet to the body of the user P. In addition to the pressing, the treatment may also include heating or cooling the body, application of vibrations (pressing in which the intensity of the pressing is varied over a short period of time), application of electromagnetic waves to the body, or irradiation with light.

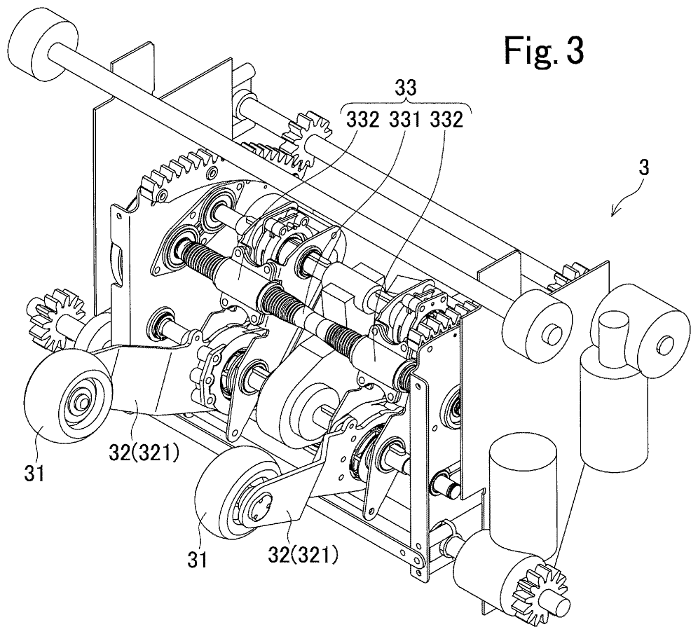

The back side treatment part 3 is configured to perform treatment using a mechanical configuration, and includes a treatment element 31, a support part 32 that physically supports the treatment element 31, and a width adjustment mechanism 33 that moves the support part 32 in the width direction. As shown in FIG. 3, the back side treatment part 3 includes a pair of treatment elements 31 arranged in the width direction. In this embodiment, two (a pair of) treatment elements 31 are provided in one back side treatment part 3 without limitation thereto, and three or more treatment elements 31 can be provided in one back side treatment part 3. The treatment element 31 is a substantially disk-shaped part, sometimes called a “kneading ball,” and performs treatment by, for example, pressing against the body of the user P.

The support part 32 is configured, as shown in FIG. 3, in order to physically support the treatment element 31. The support part 32 of this embodiment includes an elongated plate-shaped arm 321, and the treatment element 31 is provided at a leading end portion of the arm 321. As shown in FIG. 2, the treatment element 31 protrudes from the back side treatment part 3 toward the back of the neck and the back of the user P. In this embodiment, one treatment element 31 is provided on one support part 32 without limitation thereto, and a plurality of treatment elements 31 can be provided. A base end portion of the arm 321 is connected to a kneading mechanism and a tapping mechanism that are provided on the back side treatment part 3 and are actuated by a motor and a power transmission device. Driving forces generated by the kneading mechanism and the tapping mechanism are transmitted to the treatment elements 31.

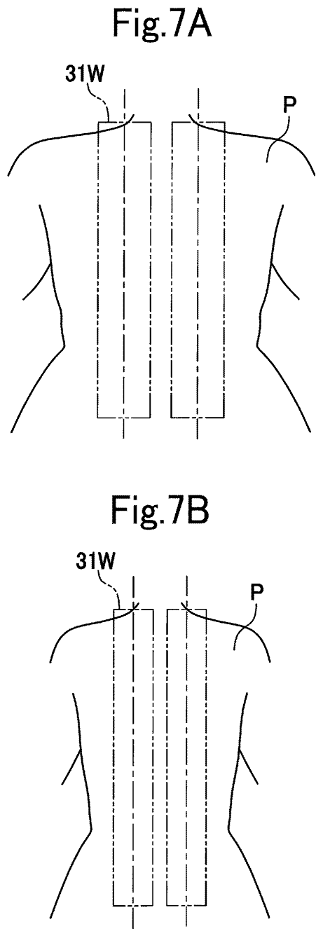

The width adjustment mechanism 33 is configured to change the distance between the pair of treatment elements 31 arranged in the width direction via the support parts 32. The width adjustment mechanism 33 includes a screw shaft 331 extending in the width direction, and a pair of guide parts 332 that are combined together by threaded engagement with the screw shaft 331. The screw shaft 331 rotates by receiving power from a width adjustment motor (not shown) provided in the back side treatment part 3. The screw shaft 331 is supported through its widthwise ends on the main body side of the back side treatment part 3 so as not to move in the axial direction when rotating. Each of the guide parts 332 is coupled with the base end portion of the corresponding arm 321. Further, each of the guide parts 332 is arranged so as to be screwed onto the screw shaft 331, and thereby moves in the width direction as the screw shaft 331 rotates. Here, the screw (more specifically, the threads) formed on the screw shaft 331 has a “reverse thread” relationship between an area corresponding to one of the pair of guide parts 332 and an area corresponding to the other one. In response to rotation of the screw shaft 331 in one direction, the pair of guide parts 332 thus move toward the opposite sides in the axial direction. The distance between the pair of guide parts 332 in the width direction can be thus increased or decreased. The rotation of the screw shaft 331 is detected by a rotary encoder (not shown), and the rotation is controlled based on the detection result. In the back side treatment part 3 configured in this manner, a treatment range 31W in which each of the treatment elements 31 actuates can be changed for each pair of the treatment elements 31 by the width adjustment mechanism 33. The treatment range 31W when the distance between the pair of the guide parts 332 in the width direction is increased is the range shown by the dashed-two dotted lines in FIG. 7A, and the treatment range 31W when the distance therebetween in the width direction is decreased is the range similarly shown in FIG. 7B. Each of the rectangles drawn by the dashed-two dotted lines roughly indicates the range within which one treatment element 31 can move. Each of the treatment elements 31 can move along a circular or wave path, for example, and the treatment range 31W is the range formed by the path.

As described above, the back side treatment part 3 can change the treatment range 31W based on the size of the user P in the width direction. In other words, the width adjustment mechanism 33 can adjust and move the treatment range 31W using the screw shaft 331 based on the size of the user P in the width direction. In this case, in addition to the above-mentioned adjustment movement, for example, as shown in FIG. 7B, when the width of the body of the user P is small, the back side treatment part 3 can perform a “kneading” action by reducing the movement along the width direction of the width adjustment mechanism 33 for each of the treatment elements 31 and combining this movement with the action of the kneading mechanism, so that the range of the kneading action in the width direction becomes narrow (for example, so that the length of the short side of the illustrated rectangle is reduced). Further, for example, as shown in FIG. 7A, when the width of the body of the user P is large, the back side treatment part 3 can perform a “kneading” action by increasing the movement along the width direction of the width adjustment mechanism 33 for each of the treatment elements 31 and combining this movement with the action of the kneading mechanism, so that the range of the kneading action in the width direction becomes wide (for example, so that the length of the short side of the illustrated rectangle is increased). The control of this “kneading” action is performed by the controller 7. In this way, the massage machine 1 determines the distance of the movement in the width direction (left and right direction) depending on the width size of the body of the user P (specifically, the width size of the back of the user P), so it is possible to perform a massage at a location appropriate to the width of the body while having the treatment range 31W adjusted to match the width of the body. In the conventional massage machines, the distance of the movement in the width direction for “kneading” is fixed, whereas the massage machine 1 of this embodiment, as described above, makes it possible to provide a treatment at a position that corresponds appropriately to the portion of the body to be treated.

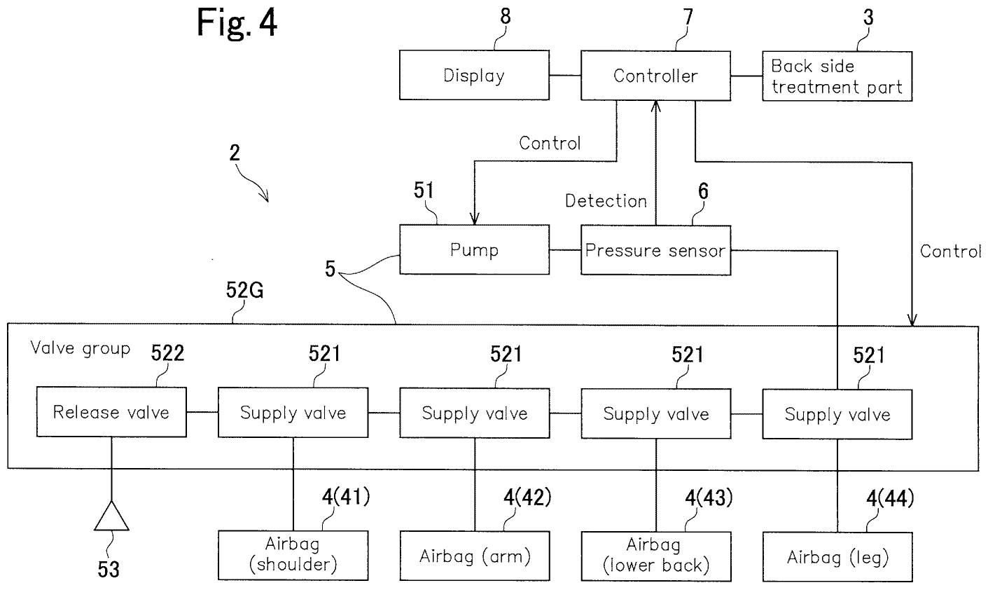

The plurality of airbags 4 are arranged at positions schematically shown in FIG. 2. The air supply mechanism 5 is provided to supply air into each of the airbags 4. The air supply mechanism 5 is composed of a pump 51, supply valves 521 as supply parts, and a discharge valve 522 as a discharge part connected to a discharge port 53 open to the atmosphere, which are controlled by the controller 7. The action of the discharge valve 522 will be described later. The supply part and the discharge part are not limited to the valves of this embodiment. As shown in FIG. 4, each of the airbags 4 is connected to the supply valve 521 for each portion to be treated of the body of the user P. The pump 51 for inflating or deflating each of the airbags 4 is connected to a valve group 52G made up of a plurality of valves 521. A pressure sensor serving as the detector 6 is connected at a position between the pump 51 and the valve group 52G. The airbag 4 combined with the detector (pressure sensor) 6 in this way can function as a lateral side abutment body X, which will be described later. In this embodiment, the pressure sensor 6 is not provided in a one-to-one relationship with each of the airbags 4, but only one pressure sensor 6 is provided at a route portion, through which the air to be supplied into the plurality of airbags 4 is converged. Such a configuration can reduce the cost of components. Even if there is only one pressure sensor 6, the first to third functions described below regarding the detection of pressure changes can be performed without any problems. There are differences regarding the fourth function, which will be described later.

The controller 7 is a microcomputer or the like built into the massage machine 1, and controls the actuation of each part of the massage machine 1. A part of the controller 7 (for example, a storage part, etc.) can be provided outside the massage machine 1 so as to be able to communicate with a main body of the controller 7 via wired or wireless communication (including an internet line).

### Description by Function

Five main functions of the massage machine 1 of this embodiment will be described below. In the following description, there are some overlapping descriptions or the same name is applied to some configurations, but they are somewhat different from each other in the configurations for executing the respective functions or functions themselves. Further, one massage machine 1 can perform all of these five functions, or can perform only one or some of the functions. It is a matter of course that the massage machine 1 can perform operations relating to functions other than these five functions.

### First Function

In the massage machine 1 of this embodiment, a first function, in which the lateral side abutment body X, the detector 6, and the controller 7 operate in cooperation with each other, will be described. The purpose of this function is to provide the massage machine 1 that can provide treatment with a content suitable for the body type of the user P among the physical conditions of the user Pin relation to the massage machine 1. The “body type” refers to the body type of the user P at the time when the user P sits on the massage machine 1, while changes over time are not taken into consideration in the first function.

The lateral side abutment body X is configured to be movable in the width direction and to be able to abut on a corresponding lateral side of the body of the user P. The physical quantity of the lateral side abutment body X changes as it moves in the width direction. Specifically, the lateral side abutment body X in this embodiment is constituted by the airbag 4 that is inflated and deflated based on the amount of air inside. In this case, the internal pressure that changes with the change in the amount of air inside the airbag 4 (the internal pressure is high when the amount of air is large and low when the amount of air is small) corresponds to the physical quantity. That is, the lateral side abutment body X in this embodiment corresponds to one or some of the plurality of airbags 4. More specifically, the lateral side abutment body X in this embodiment is one of the plurality of airbags 4 provided as described above, which is located on a corresponding lateral side of the body of the user P. Specifically, as shown in FIG. 2, the lateral side abutment body X is a shoulder airbag 41 located on each corresponding lateral side of the shoulder and/or arm of the user P. Each of the shoulder airbags 41 has its base end fixed (held) to a shoulder airbag holding frame (not shown) provided in the backrest 12, and an abutment surface of the shoulder airbag 41 moves at least in the width direction in response to inflation and deflation. Then, the shoulder airbag 41 abuts on the shoulder and/or arm of the user P under internal pressure. When detecting the body width of the user P in the range from top to bottom in the height direction of the user P, an arm airbag 42, a lower back airbag 43, etc. each can be used as the lateral side abutment body X, in addition to the shoulder airbag 41. In this case, the body of the user P can be detected by the detector 6 by sequentially activating the plurality of the lateral side abutment bodies X, which are made up of the airbags 4, for example, from top to bottom. It should be noted that the above does not exclude the case where two or more out of the shoulder airbag 41, the arm airbag 42, and the lower back airbag 43 are inflated at the same time to detect the body.

Regarding the first function, the detector 6 is disposed at a position at which it can detect a change in physical quantity that occurs based on the movement in the width direction relative to the lateral side abutment body X. That is, the detector 6 detects a change in physical quantity corresponding to the amount of the movement of the lateral side abutment body X relative to the body of the user P (the degree of inflation of the airbag 4). The abutment of a lateral side of the user P can be detected by the movement of the airbag 4 that is the lateral side abutment body (the shoulder airbag 41 in this embodiment). The detector 6 is provided so that the timing of detecting the abutment with the lateral side of the user P varies depending on the width of the body of the user P. For example, the time until the abutment occurs for the body of the user P having a large (wide) width is shorter than the time for the body having a small (narrow) width. That is, the timing of detection due to the abutment for the body of the user P having a large (wide) width is earlier than the timing of detection for the body having a small width. The detector 6 is, for example, a pressure sensor described above. The detector (pressure sensor) 6 can be provided, for example, in the middle of the piping on the “root side” near the pump 51 that supplies air into each of the plurality of airbags 4, as shown in FIG. 4. When the airbag 4 abuts on the lateral side of the user P (each corresponding lateral side of the shoulder and/or the arm), the detector 6 detects a change in the internal pressure of the airbag 4 as a change in the physical quantity. For example, in the case in which the internal pressure has increased at a constant rate and then the rate of increase becomes larger from a certain point, this corresponds to the event in which the airbag 4 abuts on the body of the user P at that point, thereby blocking the airbag 4 from expanding freely (with no load applied). Therefore, it is possible to detect abutment with the body of the user P by detecting the change in internal pressure. Here, the pressure sensor 6 is not provided individually for each of the airbags 4, so that one pressure sensor 6 detects changes in the internal pressures of the plurality of airbags 4 in a batch. Even with this configuration in which one pressure sensor 6 is provided for detections in a batch, the controller 7 controls each of the supply valves 521 that is installed in a one-to-one relationship with each of the airbags 4, so it is possible to reliably detect changes in the internal pressures of the airbags 4 that are the lateral side abutment bodies X.

The controller 7 changes the treatment range 31W of the back side treatment part 3 in the width direction based on the detection result of the detector 6. Specifically, the distance in the width direction between each pair of treatment elements 31 in an immobile state is changed by actuating the width adjusting mechanism 33. Further, the movement range in the width direction of each of a pair of treatment elements 31 (i.e., movement range of each of the treatment elements 31) can also be changed. As a result, as shown in FIG. 7A and FIG. 7B, the treatment range 31W of each of the pair of treatment elements 31 is changed.

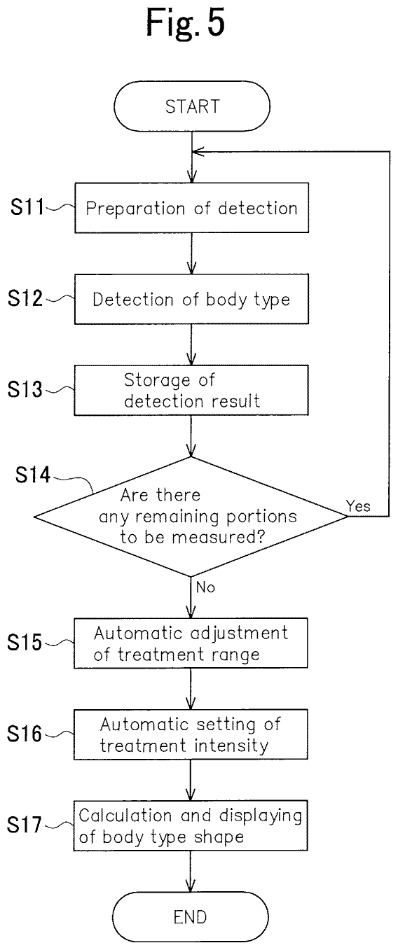

Next, an example of the procedure for detecting the body type in the first function will be described with reference to the flow diagram of FIG. 5.
(S11) A preparation operation for detecting the body type for the portion to be measured is performed. Specifically, a part, an internal pressure of which is changed for detection (the shoulder airbag 41 in this embodiment) is depressurized by, for example, letting air out, thereby making the detection start conditions constant.
(S12) Body type detection is performed by detecting a change in internal pressure. More specifically, the controller 7 opens the supply valve 521 for the portion to be measured and then actuates the pump 51. The controller 7 monitors the detection value of the detector (pressure sensor) 6. When the detection value reaches a certain change, the controller 7 calculates the body type detection result from the detection value and other information. The controller 7 estimates the body type of the user P by the calculation.
(S13) The body type detection result is stored for each portion to be measured.
(S14) In the case in which a setting is made to measure a plurality of portions, and a portion to be measured remains, S11 to S13 are repeated.
(S15) Based on the calculated body type detection result, the controller 7 activates the width adjustment mechanism 33 of the back side treatment part 3, thereby automatically adjusting the treatment range 31W of the back side treatment part 3.
(S16) The treatment intensity for each portion by the back treatment part 3 is automatically set based on the body type detection result.
(S17) The body type detection results are comprehensively evaluated to determine the shape of the body type. The determined shape of the body type is displayed (this will be explained in the section for the fifth function below).

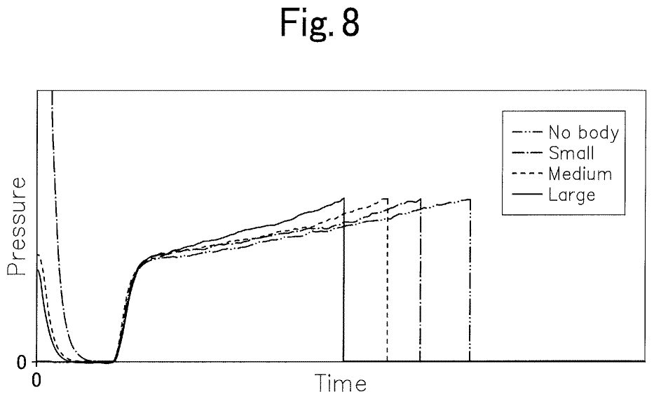

When the airbag 4 is deflated, air may not be completely released from the airbag 4, and air may remain inside. If air remains in the airbag 4 when it is deflated, the controller 7 starts detecting a change in the internal pressure with the detector (pressure sensor) 6 even when air remains in the airbag 4. However, the amount of air remaining in the airbag 4 is not necessarily constant for each of the airbags 41 to 44, and even for the same airbag 4 from time to time. This may cause variations in the initial state when the detector 6 detects pressures (see the graph lines indicating pressures at the left end (time 0) of FIG. 8). In order to deal with this, air remaining in each of the airbags 4 can be released into the atmosphere through the release port 53 by providing the release valve 522 in the valve group 52G as described above. The release valve 522 allows the massage machine 1 of this embodiment to efficiently release air remaining in the airbags 4. The controller 7, in combination with the detector 6, sets, as an initial state, a state in which no air remains in the airbag 4, so that the detected pressure value at the start of detection can be made constant (see FIG. 8, in which each line in the graph showing the pressure goes from the above-mentioned fluctuating state to 0 once). The controller 7 detects that the airbag 4 has abutted on the body of the user P when the detection values from the detector 6 reaches a certain value. More specifically, the controller 7 performs control according to the body type of the user based on the time it takes for the detection values to reach a certain value (see the upward sloping parts of the lines indicating pressures in FIG. 8). Therefore, the accuracy of pressure detection by the detector 6 (i.e., detecting that the airbag 4 has abutted on the body of the user P) is improved by suppressing the variation in the initial state of pressure detection.

According to the first function, the treatment range 31W can be changed by changing the distance between the pair of treatment elements 31 in the width direction according to the body type of the user P (specifically, based on the width of the body). Further, according to the first function, the body type of the user P can be estimated by the airbag 4 as the lateral side abutment body X in order to change the treatment range 31W.

### Second Function

Next, a second function, in which the treatment part 2, the detector 6, and the controller 7 in the massage machine 1 of this embodiment operate in association with each other, will be described.

As described above, the treatment part 2 is configured to perform treatment on the body of the user P, and is movable relative to the body of the user P and is able to abut on the same. In the second function, the treatment part 2 has the airbag 4 that inflates and deflates based on the amount of air inside. The airbag 4 that actuates in the second function is the airbag 4 that belongs to the lateral side treatment part and is located on a lateral side of the body of the user P, among the plurality of the airbags 4 provided in the massage machine 1. Specifically, the airbag 4 that actuates in the second function is an arm airbag 42 provided along the arm of the user P (this is an airbag separate from the shoulder airbag 41, which is located on the lateral side of the shoulder relating to the first function). However, it is also possible to use any one of the shoulder airbag 41, the lower back airbag 43, and the leg airbag 44 in the second function. It is also possible to use a combination of two or more of the shoulder airbag 41, the arm airbag 42, the lower back airbag 43, and the leg airbag 44 in the second function.

The detector 6 is provided to the treatment part 2 at a position at which it can detect a change in physical quantity (in this embodiment, changes in the internal pressure of the airbag 4) that occur in response to the movement relative to the body of the user P, and it detects abutment of the airbag 4 with the body of the user P. The detector 6 is provided so that the timing of detecting the abutment varies depending on the body type of the user P. When the arm airbag 42 is used, the detector 6 detects the thickness of the arm.

The controller 7 can use data on a plurality of (for example, seven levels of) treatment intensity reference values for each body type regarding the treatment intensity of the treatment part 2. The location where the data on the treatment intensity reference values is stored is not particularly limited, and can be the controller 7 itself, or a storage part provided outside the controller 7 and connected to the controller 7. The controller 7 can also be configured to acquire data on the treatment intensity reference values from outside the massage machine 1. The controller 7 sets a treatment intensity reference value based on the detection result of the detector 6 at a stage before the treatment part 2 starts treatment. For example, the controller 7 estimates the body type of the user P based on the detection result of the detector 6. The controller 7 sets one treatment intensity reference value according to the estimated body type acquired from the data of a plurality of available treatment intensity reference values. The controller 7 allows the treatment intensity to be selected based on the treatment intensity reference value (for example, the intensity can be adjusted based on the third level out of seven levels). The adjustment of the treatment intensity which has been determined as selectable can be made (increasing or decreasing the intensity from the reference treatment intensity value) by the user P operating the operation part of the massage machine 1, for example. The controller 7 actuates the treatment part 2 based on the treatment intensity reference value or the adjusted treatment intensity. Although the treatment intensity reference value is set to seven levels in this example, the present disclosure is not limited to this. The controller 7 can also set the treatment intensity reference value in a stepless manner.

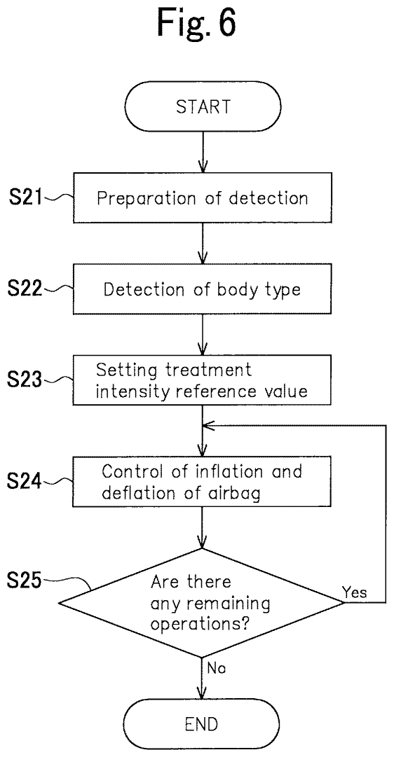

Next, an example of the procedure for detecting the body type in the second function will be described with reference to the flow diagram of FIG. 6.
(S21) A preparation operation for detecting the body type of the portion to be measured is performed. Specifically, a part, the internal pressure of which is changed for detection (arm airbag 42 in this embodiment), is depressurized by, for example, letting air out, thereby making the detection start conditions constant.
(S22) Body type detection is performed by detecting a change in internal pressure. More specifically, the controller 7 opens the supply valve 521 of the portion to be measured and then actuates the pump 51. The controller 7 monitors the detection value of the detector (pressure sensor) 6. When the detection value reaches a certain change, the controller 7 calculates the body type detection result from the detection value and other information. The controller 7 estimates the body type of the user P by the calculation.
(S23) A treatment intensity reference value is set for the treatment intensity based on the detection result.
(S24) The airbag 4 is inflated and deflated based on the set reference treatment intensity value.
(S25) If there remains any inflation and deflation operation required for the airbag(s) 4 other than the airbags 4 to which inflation and deflation operations were made in S24, S24 is repeated for the airbags 4.

This second function makes it possible to provide treatment at an appropriate intensity according to the body type of the user P (here, this corresponds to the thickness of the arm).

### Third Function

Next, a third function, in which the treatment part 2, the detector 6, and the controller 7 operate in cooperation with each other in the massage machine 1 of this embodiment, will be described. The operations performed in the third function include an operation common to the second function in terms of adjusting the treatment intensity. However, changing the treatment intensity reference value based on the detection result is not essential for the third function. The third function changes the treatment intensity set at the start of treatment by the treatment part 2 (or, an adjusted treatment intensity, if the treatment intensity has been adjusted (i.e., increased or decreased) relative to the treatment intensity reference value in the second function) depending on the posture of the user P. This third function differs from the second function in that, from the viewpoint of the timing of exerting the function and the start of treatment, detection is performed at a stage after the start of treatment. While the controller 7 estimates the body type of the user P in the second function, the controller 7 detects (estimates) the posture of the user P (more specifically, the distance between the airbag 4 used for treatment and the body of the user P) in the third function. The procedure for posture detection is basically the same as the content (detecting the body type) shown in the flow diagram of FIG. 6.

In the third function, at a stage after the start of treatment, the controller 7 actuates the treatment part 2 by changing the treatment intensity according to the detected posture of the user P, based on the detection result of the detector 6. That is, in the third function, the treatment intensity can be changed at any time during the treatment. The changing of the treatment intensity for, for example, the user P who takes a posture further away from the airbag 4 used for treatment than the posture when the treatment started makes it difficult for the inflated airbag 4 to reach the body of the user P. Therefore, the controller 7 further inflates the airbag 4 to increase the treatment intensity. On the other hand, when the user P takes a posture closer to the airbag 4 than the posture at the start of treatment, the inflated airbag 4 is easy to reach the body of the user P, so the airbag 4 is deflated to decrease the treatment intensity.

Also, the third function can be performed following the second function. In this case, for example, body type detection is performed before the start of treatment, and also body type detection is performed after the start of treatment (more precisely, posture detection of the user P (the same user) whose body type was detected before the start of treatment), and the detection results before and after the start of treatment are compared to each other. The controller 7 compares the detection result before the start of treatment with the detection result after the start of treatment, and, if there is a change between them, changes the treatment intensity of the airbag 4 (in this case, as in the second function, the treatment intensity reference value itself may be changed based on the detection results).

According to this third function, when the posture of the body of the user P changes, the abutment detection of the detector 6 also changes, so that it is possible to provide treatment with a treatment intensity that is adjusted to suit the change in the posture of the user P at any time. Further, the massage machine 1 of this embodiment is not limited to performing the third function following the second function, as described above. Thus, even if the posture of the user P changes, the massage machine 1 of this embodiment can perform treatment with a treatment intensity that is adjusted to be stronger or weaker depending on the changed posture, based on the treatment intensity adjusted (manually adjusted) by the user P before the start of treatment.

### Fourth Function

Next, a fourth function, in which the treatment part 2, the detector 6, and the controller 7 in the massage machine 1 of this embodiment operate in cooperation with each other, will be described. As an essential configuration for the fourth function, the airbag 4 serving as the treatment part(s) 2 is configured such that a plurality of airbags 4 are provided corresponding to a plurality of portions of the body of the user P. In this embodiment, the plurality of airbags 4 correspond to the shoulder airbag 41, the arm airbag 42, the lower back airbag 43, and the leg airbag 44 shown in FIG. 4, as well as airbags 4 for other portions (a buttocks airbag 45, a thigh airbag 46, etc.) shown in FIG. 2.

Regarding the fourth function, the detector 6 detects the pressures of the treatment parts 2 (airbags 4) in sequence in a time-division manner.

The controller 7 detects a change in physical quantity (a change in the internal pressure of the airbag 4 in this embodiment) using the plurality of detectors 6. Then, for an undetected portion corresponding to a detector 6 among the plurality of detectors 6 which has failed to detect the above change, the controller 7 does not actuate the treatment part 2 provided corresponding to the undetected portion. In other words, for a detected portion corresponding to a detector 6 among the plurality of detectors 6 which could detect the above change, the controller 7 actuates the treatment part 2 provided corresponding to the detected portion. For example, if the undetected portion is an arm, the controller 7 does not actuate the treatment part 2 (arm airbag 42) corresponding to the arm, and only the treatment part 2 (e.g., airbag 4 other than the arm airbag 42) corresponding to the leg, the lower back, and the shoulder (which are the detected portions by corresponding ones of the plurality of the detectors 6 which could detect changes in physical quantity) excluding the arm. It is assumed that an undetected portion is caused by the user P who is in a different seated position from the seated position assumed on design so as to have a part of the body of the user P located away from the treatment part 2 (airbag 4), making it impossible for the detector 6 to detect such a part of the body of the user P. Further, one undetected portion does not necessarily have to result from the detection by one treatment part 2 (one airbag 4), but may result from the detections by multiple treatment parts 2 (multiple airbags 4).

Further, for example, in the case in which the treatment order of a plurality of the treatment parts 2 is predetermined as a “treatment course” (this can include the case in which two or more treatment parts 2 are actuated simultaneously), the controller 7 skips a treatment for an undetected portion in the predetermined treatment order (which is set as a treatment course), and goes to the treatment preset as a subsequent treatment to the treatment for the undetected portion. Alternatively, while no treatment is performed for the undetected portion, another treatment that is not included in the predetermined treatment order (another treatment not set in the treatment course setting) is performed. The other treatment is set in advance in a storage part accessible by the controller 7, and when no treatment is performed to the undetected portion, the treatment to be performed in the preset treatment course is switched with the other treatment. In this case, the treatment preset to follow the treatment for the undetected portion is performed following the switched other treatment.

Here, in the massage machine 1 of this embodiment, for example, it is possible to perform stretching using a set of the shoulder airbag 41 and the leg airbag 44. In this case, even if the undetected portion is only one of the shoulder and the leg, stretching is hardly performed only by the actuation of the airbag 4 corresponding to the other one of the shoulder and the leg. Therefore, if one of a predetermined set (for example, the shoulder) is an undetected portion, the airbag 4 corresponding to the other one of the set (for example, the leg) can also be treated as the aforementioned “treatment part 2 provided corresponding to the undetected portion” regardless of the result of the detection by the detector 6. As a result, the “skipping” is performed based on each set mentioned above. Other examples of a preset group include a set of the arm and the lower back, and a set of the shoulder and the arm.

According to the fourth function, for example, the treatment part 2 (airbag 4) that is not in contact with the body of the user is not allowed to be actuated for a certain period of time by not providing treatment to the portion that is not detected by the detector 6. Thereby, it is possible to shorten the overall treatment time from the start to the end of the “treatment course”. Further, according to the fourth function, the massage machine 1 can concentrate on the treatment on another portion of the body of the user P, thereby improving the efficiency of treatment and enhancing the quality of treatment.

### Fifth Function

Next, a fifth function, in which the controller 7 and a display 8 operate in cooperation with each other in the massage machine 1 of this embodiment, will be described. The fifth function is served as an additional function to each of the first to fourth functions. The display 8 has, for example, a liquid crystal screen.

First, the display 8 displays an image representing message relating to particularly the first and second functions. The display 8 displays a body image that changes depending on the body type of the user P, such as a body type with a large body width (see FIG. 7A) or a body type with a small body width (see FIG. 7B). The display 8 can display more detailed classifications of the body type. The controller 7, in relation to the display 8, estimates the body type of the user P from the detection result of the detector 6, and causes the display 8 to display an image corresponding to the estimated body type. The controller 7 can retrieve one of a plurality of image patterns which are stored in advance in a storage part, or can make (generate) an image each time the image is displayed when the image is displayed on the display 8.

In particular, as a function relating to the fourth function, the controller 7 causes the display 8 to display a message relating to the undetected portion. In this case, unlike the first and second functions, the display 8 does not need to display an image, and can only display characters or symbols (e.g., messages). The message relating to the undetected portion is, for example, a message enhancing the user P to place the undetected portion at the correct position. It is a matter of course that the display 8 can also display an image relating to the correct position, in addition to the message.

According to the fifth function, in the former case, the estimated result of the body type is displayed by an image, so that the user P can easily figure out the associated information that an automatic adjustment was made for the treatment range 31W of the back side treatment part 3. In the latter case, the user P can be encouraged to take an action to make the undetected portion ready for treatment (such as changing the posture around the portion of the body corresponding to the undetected portion).

### Summary of Descriptions by Function

As described above, the massage machine 1 of this embodiment can provide treatment suited to the physical conditions of the user P by performing an operation or operations relating to one or more of the five functions. The “conditions” refer to conditions relating to the body type of the user P in the first and second functions, and to conditions relating to the posture of the body of the user P in the third and fourth functions.

### —Possibility of Changing Form—

Although the embodiment of the present disclosure has been described above, the present disclosure is not limited to the above embodiment, and various modifications are possible without departing from the gist of the present disclosure.

For example, the massage machine 1 in the above embodiment is a chair-type massage machine, but is not limited to this and can be a massage machine of various shapes and configurations. For example, the massage machine can be a bed-type massage machine in which the user lies down for use.

Further, for the back side treatment part 3, it has a pair of treatment elements 31 disposed in the width direction in the above embodiment, but the back side treatment part is not limited to this and it can also have multiple pairs of treatment elements 31. Further, the number of treatment element 31 constituting one of each pair is not limited (one in the above embodiment).

Further, the lateral side treatment part has the airbag 4 (plurality of airbags 4) in the above embodiment, but the lateral side treatment part is not limited to this, and it can have a part that is configured separately from the airbag. For example, the lateral side treatment part can have a plate-shaped portion or the like provided with a cushion (a portion other than the airbag, such as a sponge).

Further, the lateral side contact body X in the above embodiment is configured by the airbag 4 that inflates and deflates based on the amount of the inside air. However, the lateral side abutment body X does not necessarily have to be an airbag, and can be, for example, any of a variety of objects that can move relative to the lateral side of the body of the user P.

The lateral side abutment body X in the above-described embodiment is the airbag 4 provided in the treatment part 2. However, the lateral side abutment body X can be configured to be provided with no treatment function, and, instead, the detector 6 can be configured to detect abutment with the lateral side of the user P. In this case, the lateral side abutment body X is not necessarily required to perform a treatment operation.

In the above embodiment, the detection by the detector 6 is performed on the conditions that one airbag 4 (the shoulder airbag 41 in the above embodiment) abuts on the body of the user P. However, the present disclosure is not limited to this, and the conditions can be that two or more airbags 4 abut on the body of the user P.

As shown in FIG. 7A and FIG. 7B, the back side treatment part 3 of the above embodiment performs the “kneading” action with the kneading width becoming narrower or wider (or, the length of the short side of the illustrated rectangle changing) by: adjusting itself to match the width of the body of the user P; changing the movement range along the width direction of the width adjustment mechanism 33 of each treatment element 31; and combining this action with the action of the kneading mechanism. However, the present disclosure is not limited to this, and the range of movement of the width adjusting mechanism 33 of each treatment element 31 in the width direction can be fixed.

Further, the air supply mechanism 5 in the above embodiment includes the discharge valve 522. However, the air supply mechanism 5 does not necessarily have to include the discharge valve 522. In this case, in order to release the air remaining in each airbag 4, it can be configured that each supply valve 521 is opened (specifically, allowed to open naturally) for a certain time before performing the pressure detection described below, thereby discharging the air remaining in the airbag 4 through each supply valve 521.

Further, the detector 6 in the above embodiment is a pressure sensor without limitation thereto, and a piezoelectric element, a stress sensor, or the like can also be used. Depending on the type of the detector 6, it can be provided on a part that abuts on the body of the user P (for example, the surface of the airbag 4), or on a part that displaces when it abuts on the body of the user P.

Further, for the second function, the controller 7 can determine the treatment intensity of the treatment part 2 based on the detection value of the detector 6. The controller 7 respectively associates the detection values with, for example, the treatment intensities 1 to 7. More specifically, the controller 7 classifies or associates the detection values into or with respectively seven levels from level A to level G based on the magnitudes of the numerical values. The controller 7 also classifies the treatment intensities of the portions to be treated into seven levels from 1 to 7. For example, the classification is based on the relationship between treatment intensity 1 being the weakest and treatment intensity 7 being the strongest. The controller 7 can set the treatment intensity so as to be 7 when the classification of the assigned (associated) detection value is level A. In this case, the controller 7 can adjust the treatment intensity of the treatment part 2 based on the detection result of the detector 6. This allows the massage machine 1 to adjust the treatment intensity appropriately to suit the user P.

The setting of the treatment intensity is not limited to the above-mentioned setting. For example, the controller 7 can set the detection value that is the detection result of the detector 6, with a median value of treatment intensity being 4, and set each treatment intensity to a level within a total range of 7 levels (odd numbers) with 3 levels below and 3 levels above based on the median value. Incidentally, it is also possible to set the treatment intensity within a total range of an even number of levels. In this case, the controller 7 simply sets the median value so that it coincides with a boundary between two levels (for example, if a treatment intensity is set to 4 within a total range of 8 levels, there are 4 levels below and 4 levels above based on the median value). In this way, the massage machine 1 makes it easier to adjust the treatment intensity by setting a median value of the treatment intensity that serves as a reference for adjustment.

In addition, in the case of the seven-level setting mentioned above, the controller 7 can display the treatment intensity, for example, on a remote control or on a screen of a mobile terminal owned by the user P, so that the user P can set the level of the treatment intensity based on the treatment intensity 4. In this case, the controller 7 stores the median value, for example, in a storage part provided in the massage machine 1. It can be configured such that the controller 7 stores the median value in association with, for example, specific information that identifies the user. For example, when the median value stored in the storage part is called by the user P, the controller 7 sets the treatment intensity based on the median value. That is, the treatment intensity determined by the controller 7 is stored in the storage part, so that the stored treatment intensity can be used later.

Further, regarding the fourth function, it can be configured such that a plurality of the detectors 6 are provided so as to correspond to a plurality of portions of the body of the user P, at positions at which the detectors 6 can detect a change in physical quantities that occur in response to the movements of the lateral side abutment body relative to the body of the user P. For example, it can be configured such that the detector 6 is provided for each of the plurality of treatment parts 2 (airbags 4), at least for each part for which control of non-actuation regarding the fourth function is desired.

When the controller 7 performs control based on the detection of changes in internal pressure by the pressure sensor 6, it can supply air into the airbag 4 at a first flow rate as a first stage until the pressure value detected by the pressure sensor 6 reaches a certain pressure value (first pressure value). Then, when the pressure value detected by the pressure sensor 6 reaches a certain pressure value, the controller 7 can supply air into the airbag 4 at a second flow rate that is lower than the first flow rate as the second stage. Then, when the pressure value detected by the pressure sensor 6 reaches a certain pressure value (second pressure value) as a result of supplying air into the airbag 4 at the second flow rate, the controller 7 estimates the body type based on the time for which air was supplied. In this case, the time required for the first pressure value to be reached in supplying at the first flow rate is set to be constant (the same time) regardless of the body type of the user P. Therefore, the controller 7 can estimate the body type using the time during which air is supplied to the airbag 4 at the second flow rate (specifically, the time it takes for the pressure value to reach the second pressure value from the first pressure value). It can be configured such that the controller 7 estimates the body type by detecting the total time: the time during which air is supplied to the airbag 4 at the first flow rate; and the time during which air is supplied to the airbag 4 at the second flow rate. Further, it can be configured such that the controller 7 stops the supply of air into the airbag 4 after a certain time has elapsed, after the pressure value detected by the pressure sensor 6 has reached a certain value.

Further, when the detector 6 is a pressure sensor, the detection value of the pressure sensor contains noises and also contains measurement errors. To address this, the controller 7 can reduce measurement errors caused by the noise by using, for example, a low-pass filter. Further, the controller 7 can measure the offset voltage via a voltage detection device, for example, in a state where air in the airbag 4 is released (the airbag 4 is opened). Measurement errors can be reduced by performing a process to remove the offset voltage at the time of detection of the pressure.

The configuration and operation of the above embodiment will be summarized below.

(1) A massage machine 1 according to the first configuration of the above-described embodiments includes: a treatment part 2 including a lateral side abutment body X that is movable in a width direction, abuts on a lateral side of the body of a user P, and treats the lateral side of the body of the user P; a detector 6 that detects abutment of the lateral side abutment body X with the body of the user P by movement of the lateral side abutment body X; and a controller 7 that determines a treatment intensity based on a detection result of the detector 6 and actuates the treatment part 2 at the determined treatment intensity.

According to the first configuration in (1) above, the massage machine 1 can provide treatment at a treatment intensity that corresponds to the width dimension of the user P.

(2) The massage machine 1 of the first configuration in (1) above can be configured, as a second configuration, such that the controller 7 estimates a body type of the user P based on a detection result of the detector 6, sets a treatment intensity reference value according to the estimated body type, and actuates the treatment part 2 at a treatment intensity based on the treatment intensity reference value.

According to this configuration, the massage machine 1 can provide treatment performed at an appropriate treatment intensity according to the body type of the user P.

(3) The massage machine 1 according to the first configuration in (1) above can be configured, as a third configuration, such that the controller 7 actuates the treatment part 2 with changing the treatment intensity based on the detection result of the detector 6 at a stage after the start of the treatment.

According to this configuration, when the posture of the user P changes, the abutment detection by the detector 6 also changes, so that treatment can be performed with a treatment intensity that matches the posture of the user P.

(4) The massage machine 1 according to the first configuration in (1) above can be configured, as a fourth configuration, such that the controller 7 is configured not to operate the treatment part, for which the detector 6 has failed to detect abutment of the lateral side abutment body X with the body of the user P.

According to this configuration, the overall treatment time can be shortened, and treatment can be concentrated on other portions by not treating portions that do not correspond to the detection by the detector 6, resulting in more efficient treatment.

(5) The massage machine 1 according to any one of the first to fourth configurations in (1) to (4) above, can be configured, as a fifth configuration, such that the treatment part 2 includes an airbag 4 that inflates or deflates based on the amount of inside air, and the detector 6 detects a change in inner pressure when the airbag 4 abuts on the lateral side of the user P.

According to this configuration, the airbag 4 can be used to perform detection for estimating the body type.

(6) The massage machine 1 according to the second configuration in (2) above can be configured, as a six configuration, such that the massage machine 1 further includes a display 8 that displays an image, and the controller 7 estimates a body type of the user P based on the detection result of the detector 6, and allows the display 8 to display an image according to the estimated body type.

According to this configuration, the estimated result of the body type is displayed through an image, which is easy for the user P to understand the estimated result.

(7) The massage machine 1 according to the fourth configuration in (4) above can be configured, as a seventh configuration, such that the order of treatments of a plurality of the treatment parts 2 is predetermined, and the controller 7 is configured: not to actuate each of the undetected treatment parts 2 in the earliest order of treatment, when the treatment is performed in the order of treatments, and performs treatment preset as a subsequent treatment to the treatment by each of the treatment parts 2 which is not operated; or not to actuate each of the undetected treatment parts 2, and perform another treatment which is not included in the treatment order.

According to this configuration, the overall treatment time can be shortened by skipping a treatment. In addition, the quality of treatment can be improved by performing another treatment even if there is an undetected portion.

(8) The massage machine 1 according to the fifth configuration in (5) above can be configured, as an eighth configuration, such that the airbag 4 is disposed on at least one of a lateral side of a shoulder and a lateral side of an arm of the user P.

According to this configuration, the treatment range can be changed according to the body type of the user P (specifically, based on the width of the body).

(9) A massage machine 1 according to a ninth configuration of the present disclosure includes: a back side treatment part 3 that includes a treatment element 31 for treating the back of the body of the user P; a lateral side treatment part 4 including a lateral side abutment part X that is movable in a width direction and is able to abut on a lateral side of the body of the user P; a detector 6 that detects abutment with the lateral side of the user P by movement of the lateral side abutment part X; and a controller 7 that actuates the back side treatment part 3 and the lateral side treatment part 4; the back side treatment part 3 configured to change a treatment range 31W in which the treatment element 31 is actuated; the controller 7 configured to change the treatment range 31W of the back side treatment part 3 at least in the width direction, based on a detection result of the detector 6.

According to this configuration, the treatment range 31W can be changed according to the body type of the user P (specifically, based on the width of the body).

(10) The massage machine 1 according to the ninth configuration in (9) above can be configured, as a tenth configuration, such that the back side treatment element 3 includes a pair of treatment elements 31 arranged in at least in the width direction, and the distance between the pair of treatment elements 31 in the width direction is changeable, and the controller 7 is configured to change the distance in the width direction based on the detection result of the detector 6.

According to this configuration, the distance in the width direction between the pair of treatment elements 31 can be changed according to the body type of the user P (specifically, based on the width of the body).

(11) The massage machine 1 according to the fifth configuration in (5) above can include, as an eleventh configuration, a supply part 521 for supplying air into the airbag 4, and a discharge part 522 for discharging air from the airbag 4, in which the controller 7 actuates the discharge part 522 when the airbag 4 is deflated.

According to this configuration, a fluctuation in the detected pressure value at the start of detection can be prevented.

(12) The massage machine 1 according to the fifth configuration in (5) above can be configured, as a twelfth configuration, such that the controller 7 supplies air into the airbag 4 at a first flow rate and a second flow rate that is lower than the first flow rate, and the supply of air into the airbag 4 is performed at the first flow rate until a certain pressure is reached, and is performed at the second flow rate after the certain pressure is reached.

According to this configuration, the controller 7 can detect the body type by using the time during which air is supplied into the airbag 4 at the second flow rate.

(13) The massage machine 1 according to the first configuration in (1) above can be configured, as a thirteenth configuration, such that the controller 7 is able to adjust the treatment intensity based on the detection result.

According to this configuration, the treatment intensity can be adjusted appropriately to suit the user P.

(14) The massage machine 1 according to the thirteenth configuration in (13) above can be configured, as a fourteenth configuration, such that the controller 7 sets the detection result as a median value of the treatment intensity and makes the treatment intensity adjustable based on the median value.

According to this configuration, the treatment intensity can be easily adjusted by setting a median value of the treatment intensity that serves as a reference for adjustment.

As described above, according to each of the above embodiments, the treatment range can be changed according to the body type of the user P (specifically, based on the width of the body), so that treatment can be provided in such a manner to allow the physical conditions of the body of the user P to match the massage machine 1 of the user P.

The massage machine 1 of this embodiment is as described above, but the present disclosure is not limited to the above embodiment, and the design can be modified as appropriate within the intended scope of the present disclosure. Further, the effects of the present disclosure are not limited to the above-described embodiment. That is, the embodiments disclosed herein should be considered to be illustrative in all respects and not restrictive. The scope of the invention is indicated by the claims rather than by the foregoing description. Further, the scope of the present disclosure is intended to include all modifications within the meaning and scope of the claims.

What is claimed is:

1. A massage machine comprising:
a treatment part comprising a lateral side abutment body that is movable in a width direction, abuts on a lateral side of the body of a user, and treats the lateral side of the body of the user;
a detector that detects abutment of the lateral side abutment body with the body of the user by movement of the lateral side abutment body; and
a controller that determines a treatment intensity based on a detection result of the detector and actuates the treatment part at the determined treatment intensity.

2. The massage machine according to claim 1, wherein the controller estimates a body type of the user based on a detection result of the detector, sets a treatment intensity reference value according to the estimated body type, and actuates the treatment part at a treatment intensity based on the treatment intensity reference value.

3. The massage machine according to claim 1, wherein the controller actuates the treatment part with changing the treatment intensity based on the detection result of the detector at a stage after the start of the treatment.

4. The massage machine according to claim 1, wherein the controller is configured not to operate the treatment part, for which the detector has failed to detect abutment of the lateral side abutment body with the body of the user.

5. The massage machine according to claim 1, wherein the treatment part comprises an airbag that inflates or deflates based on the amount of inside air, and the detector detects a change in inner pressure when the airbag abuts on the lateral side of the user.

6. The massage machine according to claim 2, further comprising a display that displays an image, wherein the controller estimates a body type of the user based on the detection result of the detector, and allows the display to display an image according to the estimated body type.

7. The massage machine according to claim 4, wherein the order of treatments of a plurality of the treatment parts is predetermined, and the controller is configured: not to actuate each of the undetected treatment parts in the earliest order of treatment, when the treatment is performed in the order of treatments, and performs treatment preset as a subsequent treatment to the treatment by each of the treatment parts which is not operated; or not to actuate each of the undetected treatment parts, and perform another treatment which is not included in the treatment order.

8. The massage machine according to claim 5, wherein the airbag is disposed on at least one of a lateral side of a shoulder and a lateral side of an arm of the user.

9. A massage machine comprising:
a back side treatment part that comprises a treatment element for treating the back of the body of the user;
a lateral side treatment part comprising a lateral side abutment part that is movable in a width direction and is able to abut on a lateral side of the body of the user;
a detector that detects abutment with the lateral side of the user by movement of the lateral side abutment part; and
a controller that actuates the back side treatment part and the lateral side treatment part;
the back side treatment part configured to change a treatment range in which the treatment element is actuated;
the controller configured to change the treatment range of the back side treatment part at least in the width direction, based on a detection result of the detector.

10. The massage machine according to claim 9, wherein the back side treatment element comprises a pair of treatment elements arranged in at least in the width direction, and the distance between the pair of treatment elements in the width direction is changeable, and
the controller is configured to change the distance in the width direction based on the detection result of the detector.

11. The massage machine according to claim 5, further comprising:
a supply part for supplying air into the airbag, and
a discharge part for discharging air from the airbag, wherein
the controller actuates the discharge part when the airbag is deflated.

12. The massage machine according to claim 5, wherein
the controller supplies air into the airbag at a first flow rate and a second flow rate that is lower than the first flow rate, and
the supply of air into the airbag is performed at the first flow rate until a certain pressure is reached, and is performed at the second flow rate after the certain pressure is reached.

13. The massage machine according to claim 1, wherein the controller is able to adjust the treatment intensity based on the detection result.

14. The massage machine according to claim 13, wherein the controller sets the detection result as a median value of the treatment intensity and makes the treatment intensity adjustable based on the median value.
